(19) United States
(12) Patent Application Publication
(10) Pub. No.: US 2026/0256734 A1
(43) Pub. Date: Sep. 3, 2026

(54) COMPOSITIONS COMPRISING SILYMARIN (OR) CURCUMIN AND METFORMIN, METHODS AND APPLICATIONS THEREOF

(71) Applicant: ACUBIOSYS PRIVATE LIMITED, HYDERABAD (IN)

(72) Inventor: Srinivas Maddi, Hyderabad (IN)

(21) Appl. No.: 18/994,633

(22) PCT Filed: Nov. 5, 2023
(86) PCT No.: PCT/IB2023/061142

§ 371 Date: Jan. 15, 2025

(30) Foreign Application Priority Data
Nov. 8, 2022 (IN) 202241063667
Nov. 8, 2022 (IN) 202241063682

(51) Int. Cl. A61K 31/357; A61K 9/06; A61K 31/05; A61K 31/12; A61K 31/155; A61K 31/7048; A61K 47/10; A61K 47/14; A61K 47/18; A61K 47/32; A61K 47/44; A61K 47/46; A61P 17/06

(52) U.S. Cl. CPC A61K 31/357; A61K 9/06; A61K 31/05; A61K 31/12; A61K 31/155; A61K 31/7048; A61K 47/10; A61K 47/14; A61K 47/183; A61K 47/32; A61K 47/44; A61K 47/46; A61P 17/06

(57) ABSTRACT

The present disclosure generally relates to the field of pharmaceutical sciences. In particular, the disclosure relates to the compositions and kits comprising silybin (or) curcumin and biguanides or their salts. The composition further comprises therapeutically important molecules such as mangiferin and resveratrol; and oils such as lemon grass oil, and bakuchi oil and hydrogel-base-excipients mixture. The present disclosure further relates to the process for preparing said compositions and method of application in the management and treatment of skin disorders (Atopic Dermatitis, Acne, Eczema) and psoriasis.

### TECHNICAL FIELD

The present disclosure generally relates to the field of pharmaceutical sciences. In particular, the disclosure relates to the compositions and kits comprising silybin (or) curcumin and biguanides or their salts. The composition further comprises therapeutically important molecules such as mangiferin and resveratrol; oils such as lemongrass oil, and bakuchi oil and a hydrogel/cream/ointment-base-excipients mixture. The present disclosure further relates to the process for preparing said compositions and method of application in the management and treatment of skin disorders and psoriasis.

### BACKGROUND OF THE DISCLOSURE

Psoriasis is a T-cell mediated autoimmune inflammatory skin disease of hyper-activated keratinocytes affecting almost 3% global population. In 2014, WHO stated that psoriasis is a chronic, non-communicable, painful, disfiguring, and disabling disease for which there is no permanent cure. Its prevalence is known in only 19% of countries worldwide and is unequally distributed across geographical regions varying from 0.1% in Eastern Asia to 1-5% in Europe and America. In India, its prevalence is around 0.44-2.8%. Its prevalence is high in developed countries and more in adults compared to children. The disease is not only painful but also causes a negative effect on psychological well-being and detrimental quality of the life in psoriasis patients.

Psoriasis has different clinical phenotypes, chronic plaque, guttate form, erythroderma, pustular form, etc., Among them, chronic plaque or psoriasis Vulgaris is the most common form and is well-demarcated, by salmon-pink plaques in white skin (FIG. 1A) or grey plaques in black skin (FIG. 1B).

The diagnosis is usually based on the history, morphology, and distribution of skin lesions. The patient's history is required to know the potential triggers and outbreaks of psoriasis symptoms.

Skin examination along with nails, scalp, and flexures should be included. A skin biopsy may also be required to help exclude cutaneous T-cell lymphoma.

Heritability is the major risk determinant for developing psoriasis. Genome-wide association studies have identified over 80-risk loci explaining about 30% of disease heritability. The major genetic risk factor for psoriasis is HLA-C*06:02. Inheritance 5 of one allele increases the risk of psoriasis by four to five times. Further interaction with the ERAPI gene mutant, which encodes for aminopeptidase that helps to process antigens for HLA class I presentation increases the disease risk.

Mechanism of action: The expression of psoriasis is dependent on gene-environment interaction. Environmental triggers include stress, infection (particularly streptococcal), alcohol consumption, smoking, exposure to drugs such as lithium, anti-malarial, and non-steroidal inflammatory agents, and, in some cases, sunlight. Weight gain and obesity are also major risk factors for psoriasis.

During the eruption of disease, the keratinocytes multiply at much higher rates than those of non-diseased cells and fail to mature properly resulting in scaly, inflamed skin. Crucial pathways include those involving type 1 interferons and antiviral signalling, NF-κB, IL-23, and IL-17, and antigen processing and presentation by T cells. The importance of T cells in psoriasis pathogenesis involves immune cells like dendritic cells and neutrophils and non-classic immune cells such as keratinocytes. Communication between these cells happens mainly through cytokines such as 20 TNFα, IFN-γ, IL-17, and IL-22, and through activation of keratinocytes, driving epidermal hyperproliferation and production of antimicrobial proteins, growth factors, and chemokines. These factors promote the characteristic changes in psoriasis including angiogenesis, neutrophil infiltration, and increased numbers of helper T cells type 1 (Th1) and Th17 cells, creating a self-sustaining cycle of inflammation.

IL-23 and Th17 responses are considered important drivers of psoriasis. Whenever external stimulus is applied on keratinocytes release of pro-inflammatory markers such as IL-1β, IL-6, TNF-α, and INF-γ occurs leading to the activation of dendritic cells. These cells further release IL-23 and IL-12, activating Th-17 and Th1 responses. IL23 composed of p40 and p19 subunits, plays a major role in IL-17 5 biology through the maintenance and expansion of IL-17-producing immune cells (Th 17). IL-12 promotes Th1 responses, characterized by the production of IFN-γ.

IL-17 is the major source of psoriasis pathogenesis. Il-17 enhances the proliferation of Keratinocytes which in turn leads to the upregulation of CCL20, a Th17 chemoattractant, thereby setting up a positive IL-17 response feedback loop.

IFN-γ, primarily derived from Th 1 cells, was one of the first inflammatory mediators identified in psoriasis plaques. IFN-γ promotes antigen processing and expression of MHC class II molecules (such as HLA-DR) on antigen-presenting cells. In addition, IFN-γ induces the expression of proinflammatory mediators including the chemoattractant CXCL9 and CXCL10 that attract additional Th1 and cytotoxic T cells type 1 (Tc1) to the site of inflammation, thereby setting up a positive feedback loop involving interferon responses.

Treatment management: The treatment goals of plaque psoriasis are determined by various parameters: the percentage area of the body affected, a psoriasis area severity index, and impairment of quality of life assessed by a dermatology life quality index. Conventional therapy includes tropical systemic medications, phototherapy, oral systematic therapy, and immune suppressors or biologics.

If psoriasis is limited to a small area (less than 3-5% of the body surface), the mainstay of treatment is topical agents including corticosteroids, vitamin D3 analogs, calcineurin inhibitors, keratolytic, and combination topical agents (eg, corticosteroid with vitamin D3), in vehicles such as cream, ointment, foam, or gel. But the major drawback of these topical therapies is poor adherence to treatment.

Ultraviolet radiation is locally immunosuppressive. It directly affects Langerhans' cells, inhibits epidermal hyperproliferation and angiogenesis, and causes a selective reduction in cutaneous T cells via apoptosis. However, it is now used less frequently in some parts of the world due to the cumulative risk of skin cancer.

Oral systemic treatments were also a staple treatment for moderate-to-severe plaque psoriasis for some decades prior to biologics. Commonly used oral agents include methotrexate, ciclosporin, acitretin, fumarates, and apremilast. As these small molecules interact with ubiquitous intracellular targets, their actions can be relatively broad. But all these molecules in the long run have adverse side effects which include hypertension, nausea, drug-drug interactions, and many others.

Biologics have drastically changed our ability to treat psoriasis and psoriatic arthritis. They are mostly recombinant monoclonal antibodies or receptor fusion proteins and target specific inflammatory mediators. These drugs target highly specific pathways within the immune system. For example, Adalimumab, Certolizumab pegol, Etanercept, and Infliximab targets TNF, while Brodalumab, Ixekizumab, and Secukinumab targets IL-17A and Guselkumab, Risankizumab. Tildrakizumab and Ustekinumab targets IL-23. Although these biologics are more potent than other therapies these molecules also showed adverse effects in the patients which include infections in respiratory and urinary tracks, candida and tinea infections, injection site reactions, etc.

First of all, inflammation has been undoubtedly recognized as an important contributor to psoriasis. Given that the existing nonsteroidal anti-inflammatory drugs and anti-TNF drugs have shown limited utility in psoriatic patients, the novel agents with different inflammation-inhibitory mechanisms are worth pursuing. Overall, the existing therapies are synthetic, more expensive, and available only for symptomatic management. To overcome these restrictions novel formulations containing non-steroidal or natural extracts with reduced dosage, efficient targeting, and improved safety profile should be developed. Thus, there is an urgent unmet need for the development and discovery of a novel class of compositions for treating skin disorders and psoriasis.

### SUMMARY OF THE DISCLOSURE

The present invention seeks to mitigate, alleviate, circumvent, or eliminate at least one, such as one or more, of the skin disorders and psoriasis by providing a composition comprising silybin (or) curcumin and biguanides or their salts. The composition further comprises therapeutic molecule(s) such as mangiferin and resveratrol; and oils such as lemon grass oil, sandalwood oil, bakuchi oil, and hydrogel/cream/ointment base-excipient mixture.

The present invention provides a composition comprising Silybin, metformin hydrochloride, mangiferin, resveratrol, lemon grass oil, bakuchi oil, and a hydrogel/cream/ointment base-excipient mixture.

The present invention provides a composition comprising curcumin, metformin hydrochloride, mangiferin, resveratrol, lemon grass oil, bakuchi oil, and a hydrogel/cream/ointment base-excipient mixture.

The invention is directed to a method of treating psoriasis in a human being comprising topically applying the composition to areas of skin disorders and psoriasis.

Another aspect of the invention relates to such a composition for use in treating or soothing skin disorders by applying the composition to areas of the skin affected by pathological conditions. The skin disorders include Atopic dermatitis, Seborrheic dermatitis, Vitiligo, Acne, and Eczema.

Another aspect of the invention relates to such a composition for use in treating or soothing skin disorders by applying the composition to areas of the skin affected by pathological conditions.

Another aspect of the invention relates to such a composition for use in treating or soothing psoriasis lesions by applying the composition to areas of the skin affected by psoriasis.

Another aspect of the invention relates to a kit comprising Silybin, biguanides or their salts, mangiferin, resveratrol, lemon grass oil, sandalwood oil, bakuchi oil, and hydrogel/cream/ointment base-excipient mixture.

Another aspect of the invention relates to a kit comprising curcumin, biguanides or their salts, mangiferin, resveratrol, lemon grass oil, sandalwood oil, bakuchi oil, and hydrogel/cream/ointment base-excipient mixture.

Another aspect of the invention relates to a kit comprising silybin, metformin or their salts, mangiferin, resveratrol, lemon grass oil, sandalwood oil, bakuchi oil, and hydrogel/cream/ointment base-excipient mixture.

Another aspect of the invention relates to a kit comprising curcumin, metformin or their salts, mangiferin, resveratrol, lemon grass oil, sandalwood oil, bakuchi oil, and hydrogel/cream/ointment base-excipient mixture.

Another aspect of the invention relates to a kit comprising silybin, metformin or their salts, mangiferin, resveratrol, lemon grass oil, sandalwood oil, bakuchi oil, Aloe vera, Ethylenediamine tetraacetic acid (EDTA), Isopropyl alcohol (IPA), Polyethylene glycol 400 (PEG 400), Propylene glycol caprylate, crosslinked polyacrylic acid polymer and hydrogel/cream/ointment base.

Another aspect of the invention relates to a kit comprising curcumin, metformin or their salts, mangiferin, resveratrol, lemon grass oil, sandalwood oil, bakuchi oil, Aloe vera, Ethylenediamine tetraacetic acid (EDTA), Isopropyl alcohol (IPA), Polyethylene glycol 400 (PEG 400), and hydrogel/cream/ointment base.

Another aspect of the invention relates to such a kit for use in treating or soothing skin disorders and/or psoriasis lesions.

Further advantageous features of the invention are defined in the dependent claims. In addition, advantageous features of the invention are elaborated in the embodiments disclosed herein.

### BRIEF DESCRIPTION OF THE ACCOMPANYING FIGURE

In order that the disclosure may be readily understood and put into practical effect, reference will now be made to exemplary embodiments as illustrated with reference to the accompanying figures. The figures together with the detailed description below, are incorporated in and form part of the specification, and serve to further illustrate the embodiments and explain various principles and advantages, in accordance with the present disclosure where:

FIG. 1 depicts various clinical presentations of chronic plaque psoriasis. (A) Pink plaque on white skin. (B) On black skin, the plaques are grey.

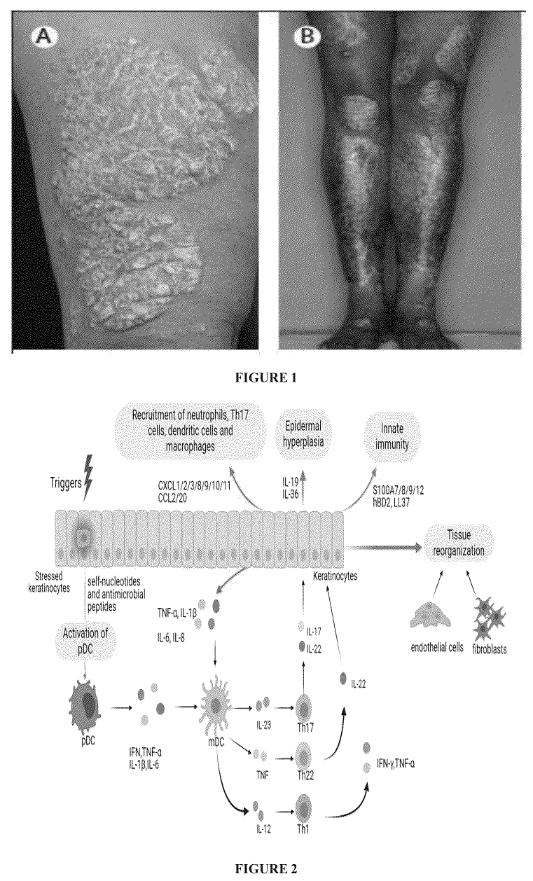

FIG. 2 depicts the mechanism of Action in psoriasis. IL-Interleukin; TNF—Tumour Necrosis Factor; mDC—Myloid Dendritic cell; pDC—Plasmacytoid dendritic cell; Th—helper T cells; INF—Interferon; CXCL—chemokine (C-X-C motif) ligand.

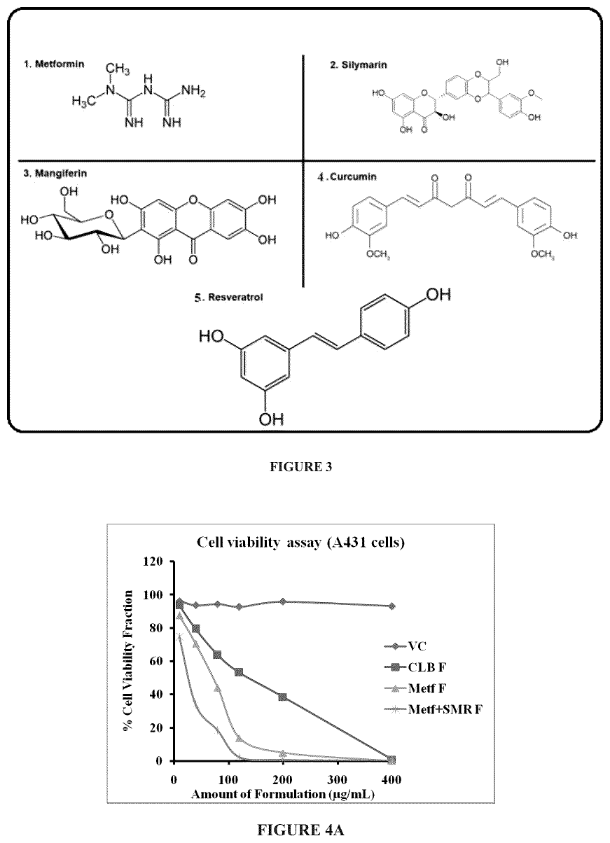

FIG. 3 depicts the chemical structures of the active drugs along with the excipients used in the formulation.

FIG. 4A depicts in vitro cytotoxicity activity of different formulations against cutaneous squamous cell carcinoma cell line (A-431). The antiproliferative/cytotoxic effect is expressed in terms of the percentage of cell viability fraction as a function of the amount of formulation (μg). VC: Vehicle control; CLB F—clobetasol formulation; Metf F: Metformin formulation; SMR: Silybin-Mangiferin-Resveratrol.

FIG. 4 B depicts in vitro cytotoxicity activity of different formulations against cutaneous squamous cell carcinoma cell line (A-431). The antiproliferative/cytotoxic effect is expressed in terms of the percentage of cell viability fraction as a function of the amount of formulation (μg). VC: Vehicle control; CLB F-clobetasol formulation; Metf F: Metformin formulation; CMR: Curcumin (0.05% w/w)-Mangiferin-Resveratrol.

FIG. 5A & FIG. 5B depicts graphs representing the mean concentrations of inflammatory markers in pg/mL. A. IL-17; B. IL-23; C. IL-1β. Data shown were mean±S.D. N=6, *P<0.05, **P<0.01, ***P<0.001 Vs Disease Control; @P<0.05, $P<0.01, #P<0.001 Vs Clobetasol using one-way ANOVA followed by Dunnett's test.

FIG. 6 depicts the protocol design for conducting in vivo anti-psoriatic efficacy evaluation of hydrogel formulations using the IMQ-induced psoriasis model.

FIG. 7A & FIG. 7B depicts the body weight changes of Metformin with SMR and CMR formulations treated IMQ-induced psoriasis model in C57BL/6 Mice

FIG. 8A & FIG. 8B depicts the changes Erythema Score of metformin with SMR and CMR formulations from In Vivo study

FIG. 9A & FIG. 9B depicts the changes of the Scale Score of metformin with SMR and CMR formulations from the In Vivo study.

FIG. 10A & FIG. 10B depicts the In vivo changes of the epidermal thickness Score of metformin with SMR and CMR formulations.

FIG. 11A & FIG. 11B depicts the cumulative Score of the vehicle and metformin with SMR and CMR formulations from Day 1 to Day 10.

FIG. 12A & FIG. 12B depicts the Spleen changes of the vehicle group and metformin with SMR and CMR formulations group

FIG. 13A & FIG. 13B depicts photomicrographs of metformin with SMR and CMR formulations treated IMQ-induced psoriasis model in C57BL/6 Mice.

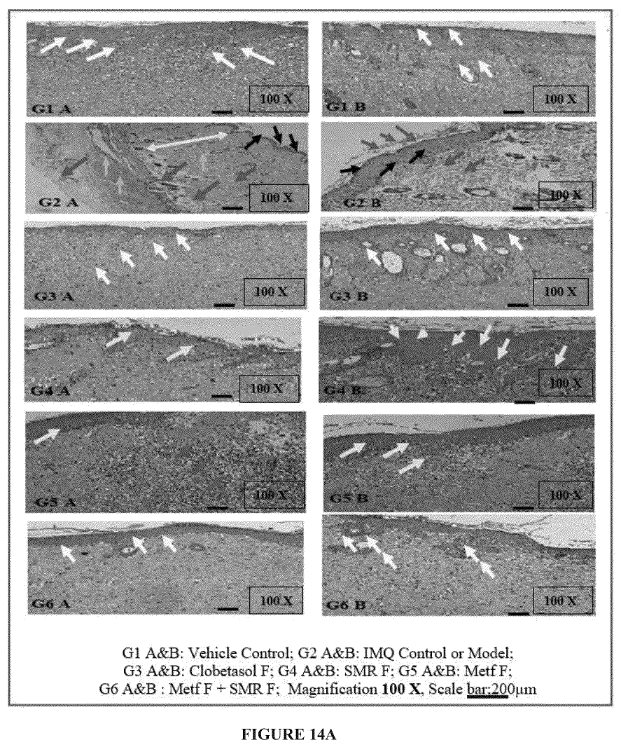

FIG. 14A & FIG. 14B depicts Histopathology changes of Metformin with SMR and CMR formulations treated IMQ-induced psoriasis model in C57BL/6 Mice.

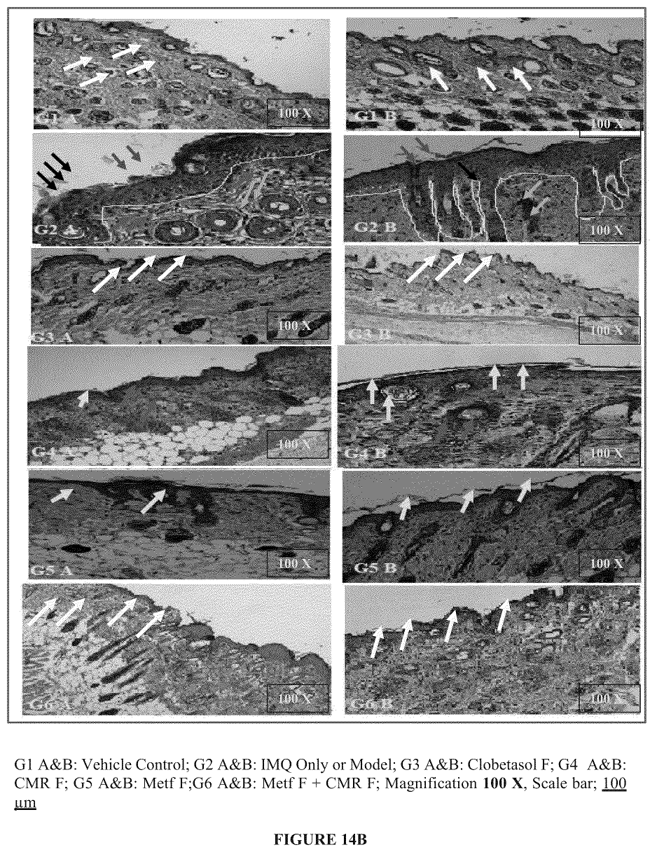

White Arrows: Normal morphology of epidermis and dermis; Blue Arrows: Acanthosis in stratum corneal layer of epidermis; Black Arrows: Hyperkeratosis and parakeratosis of stratum corneum; Red Arrows: Dermal infiltration of granulocytes and lymphocytes; Yellow Arrows: Epidermal hyperplasia; Green Arrows: Extravasation of erythrocytes in dermis;

### DESCRIPTION OF THE DISCLOSURE

The present disclosure aims to overcome the aforesaid drawbacks by providing efficient compositions for mitigating, alleviating, circumventing, or eliminating at least one, such as one or more, of the skin disorders and psoriasis by providing a composition comprising silybin and/or biguanides or their salts.

The present disclosure aims to overcome the aforesaid drawbacks by providing efficient compositions for mitigating, alleviating, circumventing, or eliminating at least one, such as one or more, of the skin disorders and psoriasis by providing a composition comprising curcumin and/or biguanides or their salts.

The present invention provides a composition comprising silybin and/or biguanide or biguanide derivatives or biguanide salts.

The present invention provides a composition comprising curcumin and/or biguanide or biguanide derivatives or biguanide salts.

In an embodiment of the present disclosure, the biguanide derivative is selected from a group comprising metformin, phenformin, or buformin, wherein biguanide salt is metformin hydrochloride. The anti-inflammatory effects of metformin are major because of the activation of AMPK and an increase in cytoplasmic AMP:ATP levels. Invivo IMQ-induced psoriatic models showed attenuation of psoriasis upon metformin administered orally.

Silybin is one of the seven flavonolignans and a major active constituent of silymarin (milk thistle seed extract). Silybin reportedly possesses strong anti-oxidant, anti-inflammation and anti-cancer properties. Silybin inhibits TNF-α-induced IL-6 and IL-1ß production, SIRT1 expression in a dose-dependent manner. Silybin reduces the proportions of IL-17-expressing T cells in CD4+ cell populations in a dose-dependent 5 manner. Silybin suppresses the expression of Th17 cell differentiation-associated genes, including IL-17 and RORY (a transcription factor for Th17 differentiation).

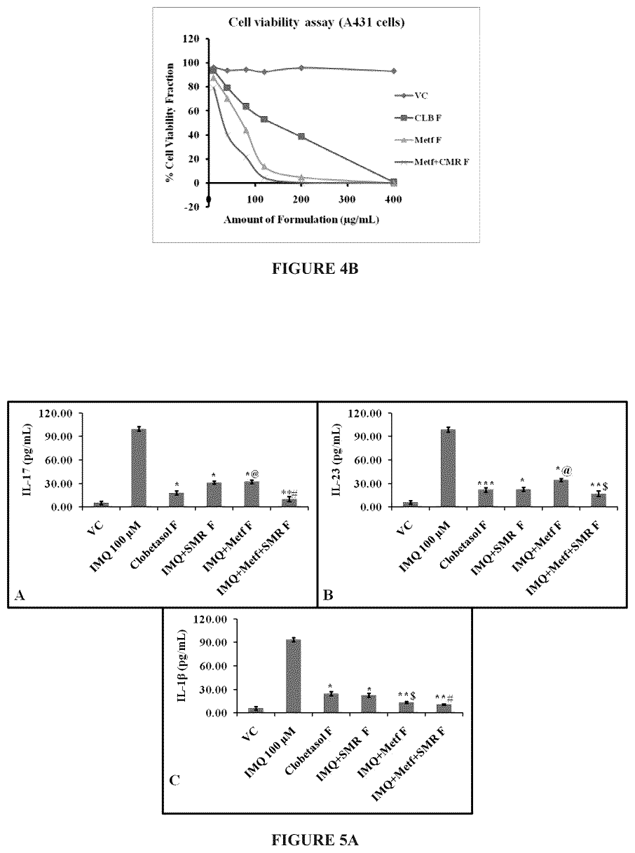

Curcumin (FIG. 3B) is a diarylheptanoid, belonging to the group of curcuminoids, which are phenolic pigments responsible for the yellow color of turmeric. Curcumin down-regulates the expression of various proinflammatory cytokines (TNF-α, IL-1, IL-2, IL-6, IL-8, and IL-12) by inactivating NF-kB. Curcumin also suppresses the 10 phosphorylation and nuclear translocation of signal transducers and activators of transcription (STATs), including STAT1, STAT3, and STAT4. Curcumin could inhibit cytokines, NF-κB and the activation STAT3, which contribute to inflammation and keratinocyte proliferation in psoriasis.

In another embodiment of the present disclosure, the composition further comprises therapeutic molecule(s) and/or oil(s) hydrogel/cream/ointment base and pharmaceutical excipient(s) and combinations thereof.

In yet another embodiment of the present disclosure, the therapeutic molecule(s) is either mangiferin or resveratrol and combinations thereof.

Mangiferin is a glucosylxanthone present in significant levels in higher plants and indifferent parts of the mango fruit, such as the peel, stalks, leaves, barks, kernel, and stone. Mangiferin prevent the production of hydroxyl radicals due to their iron chelating ability in Fenton-type reactions. In vivo psoriasis model results underlined the superior ability of mangiferin loaded glycethosomes respect to the mangiferin dispersion to promote the healing of the wound induced by TPA, confirming their potential application for the treatment of psoriasis or other skin disorders.

Resveratrol (FIG. 3D) is a stilbenoid, a type of natural phenol, and a phytoalexin produced by several plants like grapes, nuts and berries. It possesses anti-inflammatory effects in macrophage cell lines, adipocyte cell lines, cultured adipocytes and adipose tissue. Resveratrol activates Sirtuin 1 enzyme (SIRT1), which is an essential component in the fat metabolism and prevents angiogenesis of 5 adiposite tissues. Resveratrol has strong anti-inflammatory effects resulting in down regulation of NF-κB. Several studies have demonstrated that resveratrol inhibits keratinocyte proliferation while stimulating differentiation by activating SIRT1.

In yet another embodiment of the present disclosure, the oil(s) are natural extracts or synthetic volatile/non-volatile oil(s).

In still another embodiment of the present disclosure, the oil is selected from a group comprising lemon grass oil, or bakuchi oil and combinations thereof.

The present disclosure provides a composition comprising silybin, metformin hydrochloride, mangiferin, resveratrol, lemon grass oil, bakuchi oil and hydrogel/cream/ointment base.

The present disclosure provides a composition comprising curcumin, metformin hydrochloride, mangiferin, resveratrol, lemon grass oil, bakuchi oil and hydrogel/cream/ointment base.

The terms ‘gel base’ and ‘hydrogel base’ are used interchangeably in the present disclosure. The hydrogel/cream/ointment base comprises crosslinked polyacrylic acid polymer, Triethanolamine, and water.

The terms ‘gel base-excipient(s)’ and ‘hydrogel/cream/ointment base-excipient(s)’ are used interchangeably in the present disclosure.

In an embodiment of the present disclosure, the pharmaceutical excipients are selected from a group comprising thickener(s), preservative(s), alcohol(s), emollient(s), flavouring agent(s), surfactant(s), water and combinations thereof.

In an embodiment of the present disclosure,
the thickener(s) is either Aloe vera and/or crosslinked polyacrylic acid polymer;
the preservative(s) is either EDTA and/or parabens
the alcohol(s) is selected from a group comprising n-propanol, ethyl Alcohol, iso-propyl-alcohol and PEGs;
the emollient(s) is selected from a group comprising propylene glycol, lanolin, emu oil, cocoa butter, ammonium lactate and fat soluble vitamins;
the flavouring agent(s) is selected from a group comprising lemon grass oil, sandalwood oil, emu oil and bakuchi oil; and
the surfactant(s) is selected from a group comprising Propylene glycol caprylate, sodium lauryl sulphate, Cetyltrimethylammonium bromide, Spans, Tweens, N-dodecyl-N, N-dimethyl betaine, and Labrasol.

In another embodiment of the present disclosure, the composition comprises silybin, metformin hydrochloride, mangiferin, resveratrol, lemon grass oil, bakuchi oil and a hydrogel/cream/ointment base-excipient.

In another embodiment of the present disclosure, the composition comprises curcumin, metformin hydrochloride, mangiferin, resveratrol, lemon grass oil, sandalwood oil, bakuchi oil and a hydrogel/cream/ointment base-excipient.

In an embodiment of the present disclosure, the composition(s) comprises
the metformin hydrochloride at a concentration ranging from about 0.5% wt/wt to 1.5% wt/wt, preferably about 1% wt/wt;
the silybin at a concentration ranging from about 0.5% wt/wt to 1.5% wt/wt, preferably about 1% wt/wt;
the mangiferin at a concentration ranging from about 0.01% wt/wt to 0.1% wt/wt, preferably about 0.05% wt/wt;
the resveratrol at a concentration ranging from about 0.01% wt/wt to 1% wt/wt, preferably about 0.05% wt/wt;
the lemon grass oil at a concentration ranging from about 0.1% wt/wt to 1% wt/wt, preferably about 0.5% wt/wt;
the sandalwood oil at a concentration ranging from about 0.5% wt/wt to 1.5% wt/wt, preferably about 1% wt/wt;
the bakuchi oil at a concentration ranging from about 0.1% wt/wt to 0.2% wt/wt, preferably about 0.16% wt/wt; and
the gel/cream/ointment ranges at a concentration ranging about 90% wt/wt to 98% wt/wt, where in the gel base is added quantity sufficient to prepare the composition.

In an embodiment of the present disclosure, the composition(s) comprises
the metformin hydrochloride at a concentration ranging from about 0.5% wt/wt to 1.5% wt/wt, preferably about 1% wt/wt;
the curcumin at a concentration ranging from about 0.01% wt/wt to 0.1% wt/wt, preferably about 0.05% wt/wt;
the mangiferin at a concentration ranging from about 0.01% wt/wt to 0.1% wt/wt, preferably about 0.05% wt/wt;
the resveratrol at a concentration ranging from about 0.01% wt/wt to 1% wt/wt, preferably about 0.05% wt/wt;
the lemon grass oil at a concentration ranging from about 0.1% wt/wt to 1% wt/wt, preferably about 0.5% wt/wt;
the bakuchi oil at a concentration ranging from about 0.1% wt/wt to 0.2% wt/wt, preferably about 0.16% wt/wt; and
the hydrogel/cream/ointment base ranges at a concentration ranging about 90% wt/wt to 98% wt/wt, where in the gel base is added quantity sufficient to prepare the composition.

In another embodiment of the present disclosure, the total parts of the components in the composition add up to 100% w/w.

In another embodiment of the present disclosure, the composition comprises silybin at a concentration of about 1% wt/wt, metformin hydrochloride at a concentration of about 1% wt/wt, mangiferin at a concentration of about 0.05% wt/wt, resveratrol at a concentration of about 0.5% wt/wt, lemon grass oil at a concentration of about 0.5% wt/wt, bakuchi oil at a concentration of about 0.16% wt/wt and hydrogel/cream/ointment base at a concentration of about 96.79% wt/wt.

In another embodiment of the present disclosure, the composition comprises curcumin at a concentration of about 0.05% wt/wt, metformin hydrochloride at a concentration of about 1% wt/wt, mangiferin at a concentration of about 0.05% wt/wt, resveratrol at a concentration of about 0.5% wt/wt, lemon grass oil at a concentration of about 0.5% wt/wt, bakuchi oil at a concentration of about 0.16% wt/wt and gel/cream/ointment base at a concentration of about 96.74% wt/wt.

The present disclosure also provides a composition comprising
the metformin hydrochloride at a concentration ranging from about 0.5% wt/wt to 1.5% wt/wt, preferably about 1% wt/wt;
the silybin at a concentration ranging from about 0.5% wt/wt to 1.5% wt/wt, preferably about 1% wt/wt;
the mangiferin at a concentration ranging from about 0.01% wt/wt to 0.1% wt/wt, preferably about 0.05% wt/wt;
the resveratrol at a concentration ranging from about 0.1% wt/wt to 1% wt/wt, preferably about 0.5% wt/wt;
the lemon grass oil at a concentration ranging from about 0.1% wt/wt to 1% wt/wt, preferably about 0.5% wt/wt;
the sandalwood oil at a concentration ranging from about 0.5% wt/wt to 1.5% wt/wt, preferably about 1% wt/wt;
the bakuchi oil at a concentration ranging from about 0.1% wt/wt to 0.2% wt/wt, preferably about 0.16% wt/wt;
the Aloe vera is at a concentration of about 1% wt/wt;
Ethylenediamine tetraacetic acid is at a concentration of about 0.1% wt/wt;
Isopropyl alcohol at a concentration of about 10% wt/wt;
Polyethylene glycol 400 at a concentration of about 10% wt/wt;
Propylene glycol caprylate at a concentration of about 1.5% wt/wt;
Crosslinked polyacrylic acid polymer at a concentration of about 1.5% wt/wt;
the hydrogel-base at a concentration ranging about 65% wt/wt to 73% wt/wt, wherein the gel/cream/ointment base is added quantity sufficient to prepare the composition and the total parts of the components in the composition add up to 100% w/w.

The present disclosure also provides a composition comprising
the metformin hydrochloride at a concentration ranging from about 0.5% wt/wt to 1.5% wt/wt, preferably about 1% wt/wt;
the curcumin at a concentration ranging from about 0.01% wt/wt to 0.1% wt/wt, preferably about 0.05% wt/wt;
the mangiferin at a concentration ranging from about 0.01% wt/wt to 5 0.1% wt/wt, preferably about 0.05% wt/wt;
the resveratrol at a concentration ranging from about 0.1% wt/wt to 1% wt/wt, preferably about 0.5% wt/wt;
the lemon grass oil at a concentration ranging from about 0.1% wt/wt to 1% wt/wt, preferably about 0.5% wt/wt;
the sandalwood oil at a concentration ranging from about 0.5% wt/wt to 1.5% wt/wt, preferably about 1% wt/wt;
the bakuchi oil at a concentration ranging from about 0.1% wt/wt to 0.2% wt/wt; preferably about 0.16% wt/wt;
the Aloe vera is at a concentration of about 1% wt/wt;
Ethylenediamine tetraacetic acid is at a concentration of about 0.1% wt/wt;
Isopropyl alcohol at a concentration of about 10% wt/wt;
Polyethylene glycol 400 at a concentration of about 10% wt/wt;
Propylene glycol caprylate at a concentration of about 1.5% wt/wt;
Crosslinked polyacrylic acid polymer at a concentration of about 1.5% wt/wt;
the hydrogel/cream/ointment-base at a concentration ranging about 65% wt/wt to 73% wt/wt, wherein the gel/cream/ointment base is added quantity sufficient to prepare the composition and the total parts of the components in the composition add up to100% w/w.

The present disclosure provides a process for preparing the composition comprising metformin formulation, curcumin formulation and metformin-curcumin formulation.

The present disclosure provides a process for preparing the composition comprising metformin-silybin formulation.

The present disclosure also provides a composition comprising silybin at a concentration of about 1% wt/wt, metformin hydrochloride at a concentration of about 1% wt/wt, mangiferin at a concentration of about 0.05% wt/wt, resveratrol at a concentration of about 0.5% wt/wt, lemon grass oil at a concentration of about 0.5% wt/wt, bakuchi oil at a concentration of about 0.16% wt/wt, the Aloe vera is at a concentration of about 1% wt/wt, Ethylenediamine tetraacetic acid is at a concentration of about 0.1% wt/wt, Polyethylene glycol 400 at a concentration of about 10% wt/wt, Propylene glycol caprylate at a concentration of about 1.5% wt/wt, crosslinked polyacrylic acid polymer at a concentration of about 1.5% wt/wt, and the hydrogel/cream/ointment base-excipients mixture at a concentration of about 81.79% wt/wt, and the total parts of the components in the composition add up to 100% w/w.

The present disclosure also provides a composition comprising curcumin at a concentration of about 0.05% wt/wt, metformin hydrochloride at a concentration of about 1% wt/wt, mangiferin at a concentration of about 0.05% wt/wt, resveratrol at a concentration of about 0.5% wt/wt, lemon grass oil at a concentration of about 0.5% wt/wt, bakuchi oil at a concentration of about 0.16% wt/wt, the Aloe vera is at a concentration of about 1% wt/wt, Ethylenediamine tetraacetic acid is at a concentration of about 0.1% wt/wt, Polyethylene glycol 400 at a concentration of about 10% wt/wt, Propylene glycol caprylate at a concentration of about 1.5% wt/wt, crosslinked polyacrylic acid polymer at a concentration of about 1.5% wt/wt, and the hydrogel/cream/ointment base-excipients mixture at a concentration of about 82.64% wt/wt, and the total parts of the components in the composition add up to 100% w/w.

In an embodiment of the present disclosure, hydro gel was prepared by slowly dispersing crosslinked polyacrylic acid polymer (1.5% w/v) in water under continuous stirring. After settling, the dispersion was neutralized by adding Triethanolamine to obtain gel structure (hydrogel base).

In an embodiment of the present disclosure, hydro gel-excipients mixture was prepared by adding different excipients with varying ratios to the gel-base. The excipients and oils were accurately weighed and added to the gel-base in the following order: thickeners—Aloe vera (1% w/w), preservatives—EDTA (0.1% w/w) and emollients-propylene glycol (10% v/w), flavouring agents-lemon grass oil (0.5% v/w), (1% v/w), and bakuchi oil (0.16% v/w) and surfactant-Propylene glycol caprylate (1.5% v/w). The mixture was kept under continuous stirring until it becomes homogenous.

### Preparation of API Formulations (Metf+SMR Formulation)

Accurately weighed metformin (1% w/w), silybin (1% w/w), mangiferin (0.05% w/w), and resveratrol (0.5% w/w) were added to the hydrogel/cream/ointment-excipient mixture and stirred in an overhead stirrer till it becomes a homogenous formulation.

### Preparation of API Formulations (Metf+CMR Formulation)

Accurately weighed metformin (1% w/w), curcumin (0.05% w/w), mangiferin (0.05% w/w), and resveratrol (0.5% w/w) were added to the hydrogel/cream/ointment-excipient mixture and stirred in an overhead stirrer till it becomes a homogenous formulation.

### EXAMPLES

### Example 1

### Compositions of Silybin and Metformin—Formulation-1:

Formulation 1 is also called as silybin, Metformin, Mangiferin-Resveratrol formulation. Composition comprises the following ingredients:

TABLE 1
Compositions of silybin and Metformin - formulation-1

| S. | | Composition |
|---|---|---|
| No | API/ Excipient | (% w/w) |
| 1. | Metformin HCL | 1 |
| 2. | silybin | 1 |
| 3. | Mangiferin | 0.05 |
| 4. | Resveratrol | 0.5 |
| 5. | Aloe vera | 1 |
| 6. | EDTA | 0.1 |
| 7. | PEG 400 | 10 |
| 8. | Lemon grass oil | 0.5 |
| 9. | Bakuchi Oil | 0.16 |
| 10. | Propylene glycol caprylate | 1.5 |
| 11. | Crosslinked polyacrylic acid polymer | 1.5 |
| 12. | Gel/cream/ointment base | q.s |

### Example 2

### Compositions of Curcumin and Metformin—Formulation-1:

Formulation 1 is also called as Curcumin, Metformin, Mangiferin-Resveratrol formulation. Composition comprises the following ingredients:

TABLE 1
Compositions of Silybin and Metformin - formulation-1

| S. | | Composition |
|---|---|---|
| No | API/ Excipient | (% w/w) |
| 1. | Metformin HCL | 1 |
| 2. | Curcumin | 0.05 |
| 3. | Mangiferin | 0.05 |
| 4. | Resveratrol | 0.5 |
| 5. | Aloe vera | 1 |
| 6. | EDTA | 0.1 |
| 7. | PEG 400 | 10 |
| 8. | Lemon grass oil | 0.5 |
| 9. | Bakuchi Oil | 0.16 |
| 10. | Propylene glycol caprylate | 1.5 |
| 11. | Crosslinked polyacrylic acid polymer | 1.5 |
| 12. | Gel/cream/ointment base | q.s |

### Example 3

### Compositions of Silybin and Metformin—Formulation-2:

Formulation 2 is also called as silybin, Metformin, Mangiferin-Resveratrol formulation. Composition comprises the following ingredients:

TABLE 2
Compositions of Silybin and Metformin - formulation-2

| S. | | Composition |
|---|---|---|
| No | API/ Excipient | (% w/w) |
| 1. | Metformin HCL | 0.5 |
| 2. | silybin | 0.5 |
| 3. | Mangiferin | 0.01 |
| 4. | Resveratrol | 0.1 |
| 5. | Aloe vera | 1 |
| 6. | EDTA | 0.1 |
| 7. | PEG 400 | 10 |
| 8. | Lemon grass oil | 0.1 |
| 9. | Bakuchi Oil | 0.16 |
| 10. | Propylene glycol caprylate | 1.5 |
| 11. | crosslinked polyacrylic acid | 1.5 |
| | polymer having viscosity 940 | |
| 12. | Gel/cream/ointment base | q.s |

### Example 4

### Compositions of Curcumin and Metformin—Formulation-2:

Formulation 2 is also called as Curcumin, Metformin, Mangiferin-Resveratrol formulation. Composition comprises the following ingredients:

TABLE 2
Compositions of Curcumin and Metformin - formulation-2

| S. | | Composition |
|---|---|---|
| No | API/ Excipient | (% w/w) |
| 1. | Metformin HCL | 0.5 |
| 2. | Curcumin | 0.01 |
| 3. | Mangiferin | 0.01 |
| 4. | Resveratrol | 0.1 |
| 5. | Aloe vera | 1 |
| 6. | EDTA | 0.1 |
| 7. | PEG 400 | 10 |
| 8. | Lemon grass oil | 0.1 |
| 9. | Bakuchi Oil | 0.1 |
| 10. | Propylene glycol caprylate | 1.5 |
| 11. | Crosslinked polyacrylic acid polymer | 1.5 |
| 12. | Gel/cream/ointment base | q.s |

### Example 5

### Compositions of Silybin and Metformin—Formulation-3:

Formulation 3 is also called as silybin, Metformin, Mangiferin-Resveratrol formulation. Composition comprises the following ingredients:

TABLE 3
Compositions of silybin and Metformin - formulation-3

| S. | | Composition |
|---|---|---|
| No | API/ Excipient | (% w/w) |
| 1. | Metformin HCL | 1.5 |
| 2. | silybin | 1.5 |
| 3. | Mangiferin | 0.1 |
| 4. | Resveratrol | 0.1 |
| 5. | Aloe vera | 1 |
| 6. | EDTA | 0.1 |
| 7. | PEG 400 | 10 |
| 8. | Lemon grass oil | 1 |
| 9. | Bakuchi Oil | 0.2 |
| 10. | Propylene glycol caprylate | 1.5 |
| 11. | Crosslinked polyacrylic acid polymer | 1.5 |
| 12. | Gel/cream/ointment base | q.s |

### Example 6

### Compositions of Curcumin and Metformin—Formulation-3:

Formulation 3 is also called as Curcumin, Metformin, Mangiferin-Resveratrol formulation. Composition comprises the following ingredients:

TABLE 4
Compositions of Curcumin and Metformin - formulation-3

| S. | | Composition |
|---|---|---|
| No | API/ Excipient | (% w/w) |
| 1. | Metformin HCL | 1.5 |
| 2. | Curcumin | 0.1 |
| 3. | Mangiferin | 0.1 |
| 4. | Resveratrol | 1 |
| 5. | Aloe vera | 1 |
| 6. | EDTA | 0.1 |
| 7. | PEG 400 | 10 |
| 8. | Lemon grass oil | 1 |
| 9. | Bakuchi Oil | 0.2 |
| 10. | Propylene glycol caprylate | 1.5 |
| 11. | Crosslinked polyacrylic acid | 1.5 |
| | polymer having viscosity 940 | |
| 12. | Gel/cream/ointment base | q.s |

### Example 7

### Formulation-1 Preparation:

### Preparation of Hydrogel/Cream/Ointment-Base

1. The hydrogel/cream/ointment-base was prepared by slowly dispersing crosslinked polyacrylic acid polymer (1.5% w/v) in water under continuous stirring.
2. After settling, the dispersion was neutralized by adding Triethanolamine to obtain gel structure.

### Preparation of Hydrogel/Cream/Ointment—Excipients Mixture

3. The hydrogel/cream/ointment-excipients mixture was prepared by adding different excipients with varying ratios to the gel-base.
4. The excipients were accurately weighed and added to the gel-base in the following order: thickeners—Aloe vera (1% w/w), preservatives—EDTA (0.1% w/w) and emollients—propylene glycol (10% v/w), flavouring agents—lemon grass oil (0.5% v/w), and bakuchi oil (0.16% v/w) and surfactant—Propylene glycol caprylate (1.5% v/w).
5. The mixture was kept under continuous stirring until it becomes homogenous.

### Preparation of API Formulations—

6. Metf+SMR formulation: accurately weighed metformin (1% w/w), silybin (1% w/w), mangiferin (0.05% w/w), and resveratrol (0.5% w/w) were added to the hydrogel-excipient mixture and stirred in an overhead stirrer till it becomes a homogenous formulation.
7. Metf+CMR formulation: accurately weighed metformin (1% w/w), curcumin (0.05% w/w), mangiferin (0.05% w/w), and resveratrol (0.5% w/w) were added to the hydrogel-excipient mixture and stirred in an overhead stirrer till it becomes a homogenous formulation.

### Example 8

### Biological Activity Studies:

### In Vitro Studies:

Experimental data show that imiquimod (IMQ)-induced dermatitis in mice closely resembles human psoriasis lesions not only with regard to phenotypic and histological characteristics but also in the development of the lesions, which is based on the IL-23/IL-17A axis. This study was designed to investigate the effect of a Metf+SMR and Metf+SMR hydro-gel formulation on an IMQ-induced psoriasis-like mouse model. The composition of the hydrogel formulation prepared by using 1.5% crosslinked polyacrylic acid polymer and 1.5% triethanolamine is provided. In doing so, the Applicant expected to obtain supportive evidence of a role for the formulation in treating psoriasis. For that the Applicant has developed hydrogel-based formulations: 1. Metformin and 2. Metformin-silybin (Metf+SMR) 3. Metformin-curcumin (Metf+CMR) and studied invitro cell toxicity in A431 cells by MTT assay and invitro IMQ-induced psoriatic modal using HaCaT cells and determined the inflammatory markers (IL-1β, IL-23 and IL-17).

### Cytotoxicity Assay

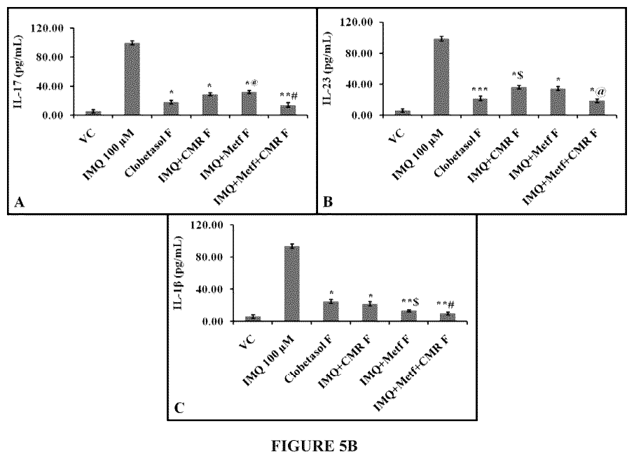

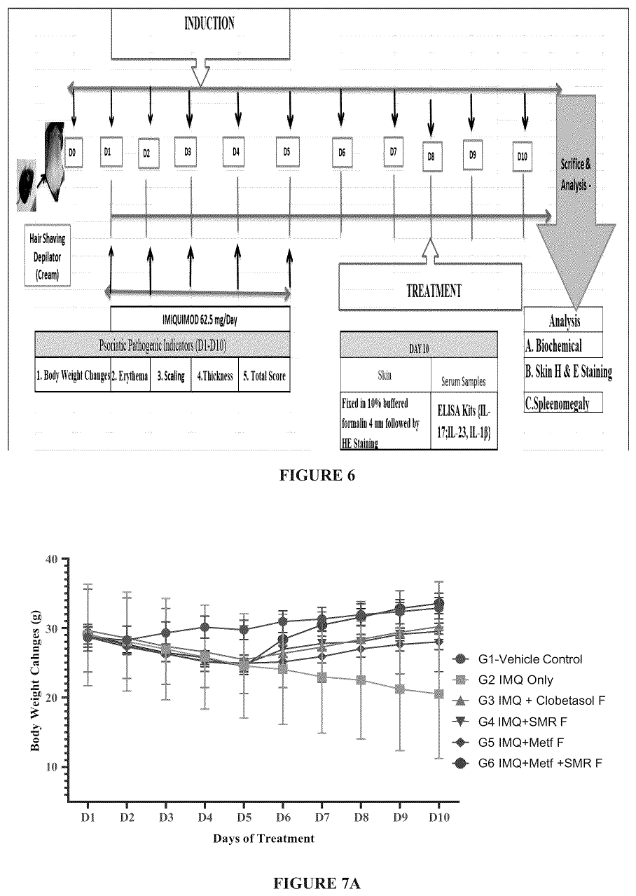

The formulation concentration ranged from 10 to 400 μg, and significantly decreased viability of A-431 cells when treated with our novel formulations compared to that of clobetasol. However, formulation Metf+SMR and Metf+CMR showed strong cytotoxicity at the concentration range of <40 μg (FIG. 4A & 4B). The IC50 values of the various formulations used in the study are given in Table 5 below.

TABLE 5
IC50 values of different formulations

| Formulation | IC50 (μg) |
|---|---|
| VC | — |
| CLB F | 142.7 |
| Metf F | 75.4 |
| Metf + SMR F | 22.8 |
| Metf + CMR F | 25.3 |

### Invitro IMQ-Induced Psoriatic Model

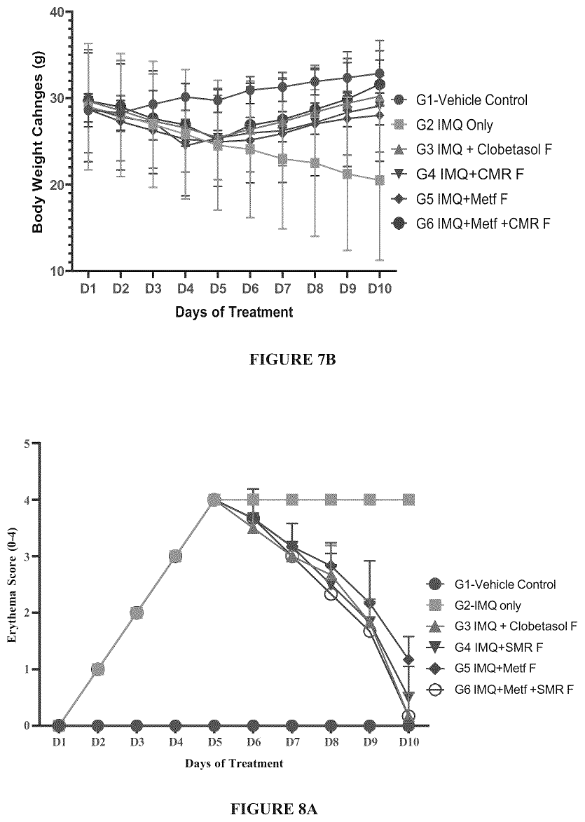

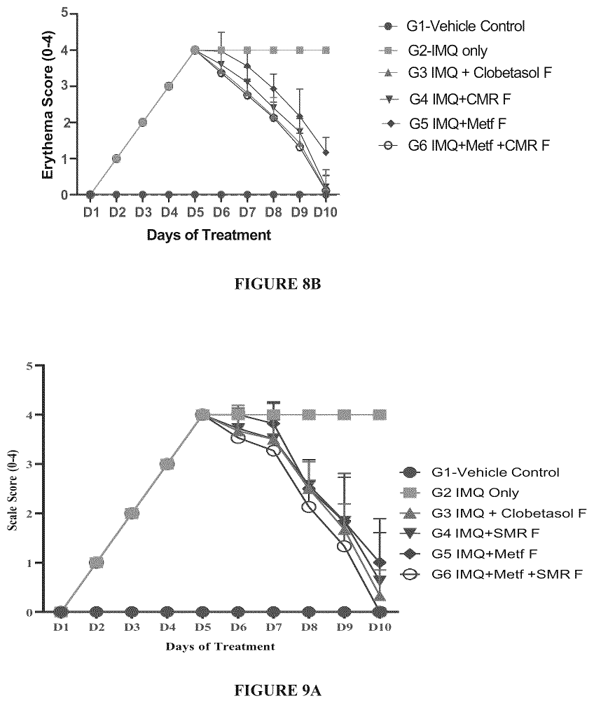

To set up an efficient and consistent model for psoriasis in vitro, differentiated HaCaT cells were used to screen our test formulations. HaCaT cells were incubated with 2 mM calcium chloride (CaCl2) to achieve differentiation. Induction of psoriatic-like inflammation was achieved by treating the cells with Imiquimod (IMQ) (100 5 μM). The differentiation and inflammation were confirmed by microscopic images. After inflammation, the cells were treated with different formulation dissolved in culture media and incubated for 48 hr. Clobetasol cream formulation was used as a positive control. After incubation, the conditioned media was collected and used for estimation of inflammatory markers (IL-17, IL-1β and IL-23). Our results showed 10 that upon IMQ treatment the concentration of inflammatory markers was significantly increased (FIG. 5). And upon treatment with formulations, their concentrations reduced significantly. The highest significant reduction was observed in Metf+SMR and Metf+CMR formulation when compared to other formulations (FIG. 6).

TABLE 6
Mean concentrations of inflammatory markers

| Group | IL-17 (pg/mL) | IL-23 (pg/mL) | IL-1β (pg/mL) |
|---|---|---|---|
| VC | 5.16 ± 2.28 | 6.07 ± 2.28 | 6.14 ± 2.25 |
| IMQ 100 μM | 99.82 ± 3.03 | 98.73 ± 3.27 | 93.63 ± 2.87 |
| Clobetasol F | 18.06 ± 2.47 | 21.74 ± 3.02 | 24.82 ± 2.62 |
| IMQ + SMR F | 31.09 ± 2.19 | 22.51 ± 2.58 | 22.54 ± 2.67 |
| IMQ + Metf F | 32.15 ± 2.16 | 34.49 ± 2.18 | 13.27 ± 1.33 |
| IMQ + Metf + SMR F | 10.17 ± 3.09 | 16.75 ± 3.64 | 10.8 ± 0.67 |
| IMQ + Metf + CMR F | 14.05 ± 3.06 | 18.62 ± 2.50 | 09.75 ± 1.67 |

R: Resveratrol;
Met F: Metformin HCL
IMQ: Imiquimod

### IMQ Induced Psoriatic Plaque Model

Hair was shaved along the back of the anesthetized mouse from shoulder to hip by using electric clippers or depilatory cream. Psoriasis-like lesions were induced by a daily topical application of a commercially available IMQ cream (62.5 mg/day for 5 days D1-D5) on the shaved back skin of C57BL/6 mice.

Animals were randomly assigned to the different groups after reaching a maximum score of 4 with respect to erythema, scale, and thickness. Animals that didn't score 4 were excluded from the experiment. Sham psoriasis animals were administered with Vaseline cream; IMQ animals, were only challenged with imiquimod cream, and IMQ animals were treated with a hydrogel that was topically applied using a nylon mesh for even distribution of the hydrogel, for the whole duration of the experiment.

### Scoring Severity of Skin Inflammation

To score the severity of inflammation of the back skin, an objective scoring system was developed based on the clinical Psoriasis Area and severity index (PASI), except that for the mouse model the affected skin area is not taken into account in the overall score. Erythema, scaling, and thickening was scored independently on a scale from 0 to 4:0, none; 1, slight; 2, moderate; 3, marked; 4, very marked. The level of erythema was scored using a scoring table with red taints. The cumulative score (erythema plus scaling plus thickening) served as a measure of the severity of inflammation (scale 0-12).

### PASI Score;

0: None; 1: Slight; 2: Moderate; 3: Marked; 4: Very Marked.

### Body Weight Studies:

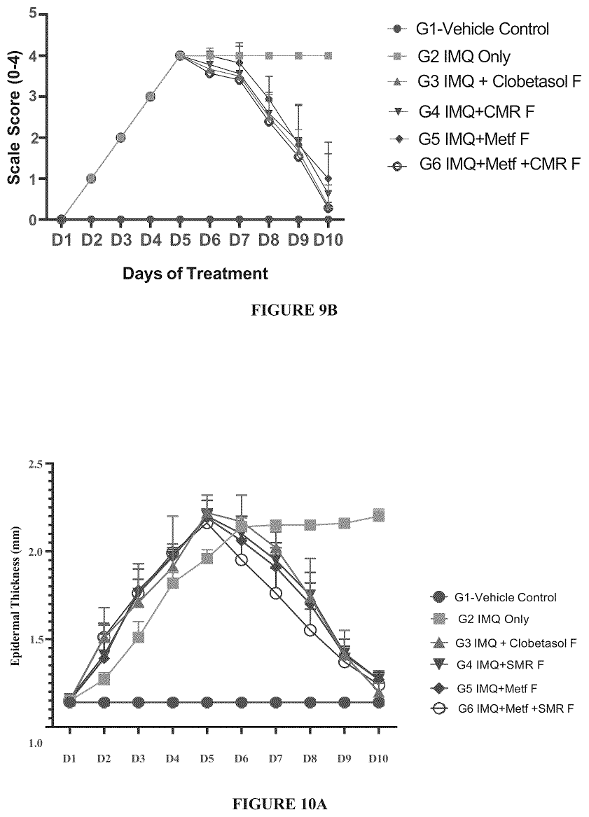

Data were shown (in FIG. 7A & 7B) Mean±S.D. N=6, *P<0.05, **P<0.01, ***P<0.001 Vs Disease Control; @P<0.05,$P<0.01, #P<0.001 Vs Clobetasol using one way ANOVA followed by Dunnett's test.

The body weight reduction was observed across all groups during the disease 5 induction phase from Day 1 to Day 5 except G1 (vehicle control). Significant weight gain was observed with G4 and G5 i.e. SMR F (P<0.05) and Met F ((P<0.05) treated groups compared to disease control and clobetasol. The Metf+SMR F and Metf+CMR F formulation resulted in significant weight gain (p<0.001) compared to clobetasol

### Erythema Studies:

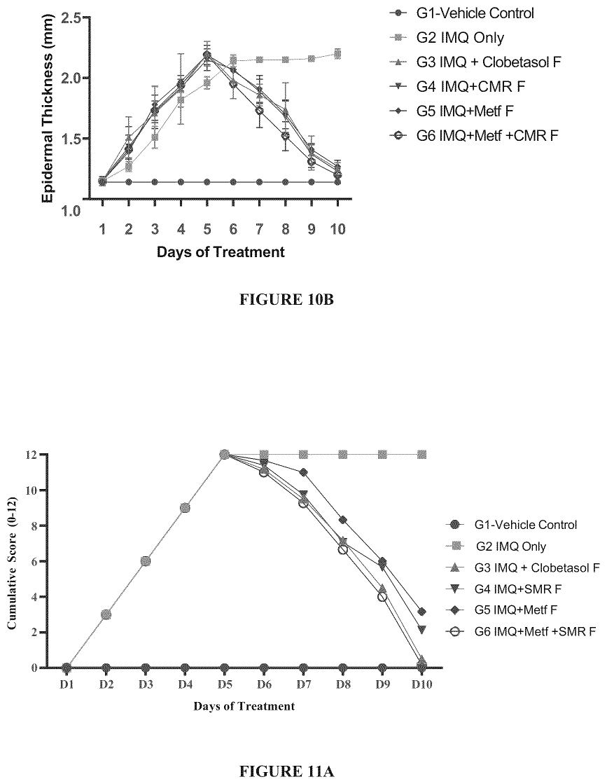

Data were shown (in FIG. 8A & 8B) Mean±S.D. N=6, *P<0.05, **P<0.01, ***P<0.001 Vs Disease Control using one-way ANOVA followed by Dunnett's test.

Zero scale erythema was observed in the G1-vehicle group, i.e., animals were healthy without any psoriatic symptoms. All the formulations treated showed the highest erythema scale of 4 on day 5. The erythema score of the SMR and CMR formulation group and Met F treated groups came down from 4 to 0.5 and 0.55, respectively. The G7 and G8 (Metf+SMR F/CMR F treated) groups reduced erythema from 4 to 0.17 significantly (p<0.001) compared to disease control. The Metf+SMR F/CMR F, a combination would be a potent anti-erythematic compared to their individual applications.

### Skin Scaling Studies:

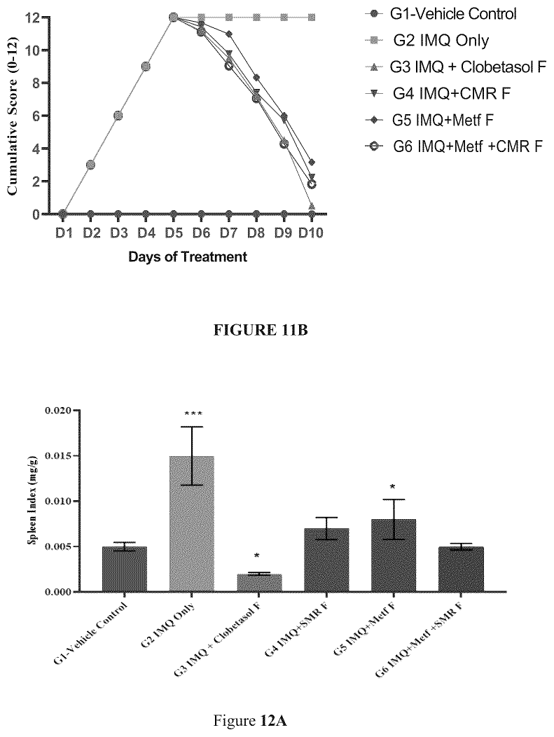

Data were shown (in FIG. 9A & 9B) Mean±S.D. N=6, *P<0.05, **P<0.01, ***P<0.001 Vs Disease Control using one-way ANOVA followed by Dunnett's test.

Psoriatic scale scores were found to be 4 across all groups on day except vehicle control. A significant (p<0.05) scale reduction was observed from 4 to 0.63 and 0.98 for SMR F/CMR-F (G4, G5) and Metf-G6 treated animals. The G7 and G 8-Metf+SMR F/CMR F received group was characterized by high significant (p<0.001) reduction of scale score from 4 to zero (0) as shown in FIG. 9. It acts a more potent when compared to clobetasol Formulation group.

### Epidermal Thickness Studies:

One of the important characteristic feature of psoriasis and the psoriasis-like skin inflammation was abnormal increase of epidermal skin thickness which was measured by vernier calipers.

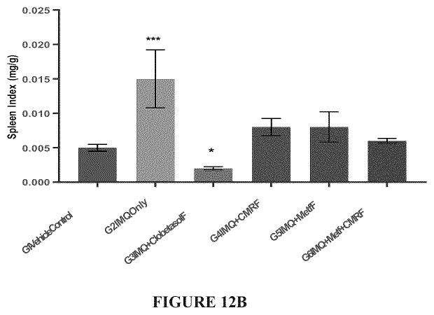

Data were shown (in FIG. 10A & 10B) Mean±S.D. N=6, *P<0.05, **P<0.01, ***P<0.001 Vs Disease Control using one-way ANOVA followed by Dunnett's test.

The epidermal skin thickness was increased drastically from 1.15 mm to 2.22 mm with IMQ only (63.5 mg/kg/day) topically applied group. The skin thickness was increased across all groups upon IMQ application from day 1 to day 5 except vehicle control. The thickness of SMR F/CMR F and Metf F treated groups was found to be 1.32 and 1.27 mm on day 10. A high significant (P<0.001) epidermal thickness reduction was observed with Metf+SMR F and Metf+SMR F compared to disease control (2.20 mm).

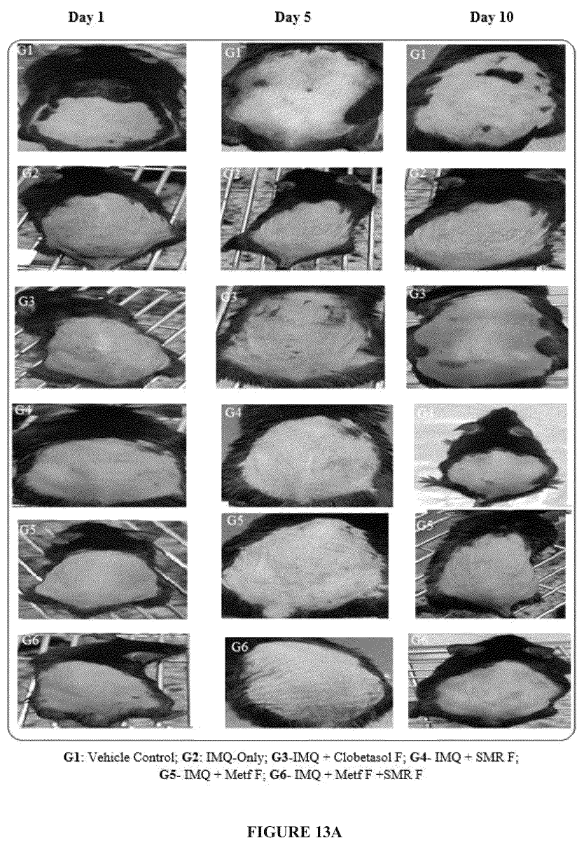

Cumulative Score results (erythema+scaling+skin thickness) served as a measure of the severity of inflammation (scale 0-12). Data were shown (in FIG. 11A & 11B) Mean±S.D. N=6, *P<0.05, **P<0.01, ***P<0.001 Vs Disease Control using one-way ANOVA followed by Dunnett's test.

The cumulative score is the sum of erythema (4)+Scale (4)+skin thickness (4) is equivalent to 12. The IMQ only received animals cumulative score was found to be 12 on day 5. Upon receiving SMR F/CMR F treatment, a significant (p<0.05) reduction was observed for both formulations from day 6 to day 10. The more significant (p<0.001) reduction of cumulative score of 0.17 on day 10 with G6-Metf F+SMR F was reported, whereas the more significant (p<0.001) reduction of cumulative score of 1.82 on day 10 with G6-Metf F+CMR F was reported. The cumulative score reduction was significantly high in case of SMR F and CMR F compared to G2 (12) on day 10.

### Spleen Index Studies:

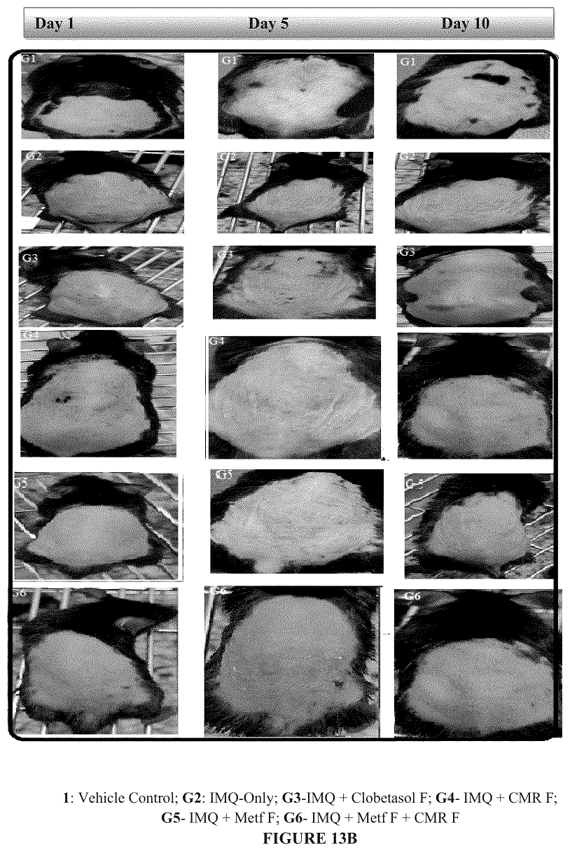

Data were shown (in FIG. 12A & 12B) Mean±S.D. N=6, *P<0.05, **P<0.01, 5 ***P<0.001 Vs Disease Control using one-way ANOVA followed by Dunnett's test.

The abnormal increase in spleen size was reported with clobetasol treatment (D6-D10) whereas as SMR F and Metf+SMR F/CMR F resulted in normal spleen size (0.005 mg/g) as shown in FIG. 12. The G2-IMQ group spleen size was increased to 0.015 mg/g. G3-clobetasol treated group spleen size was 0.02 mg/g. So the combination of G6-Metf+SMR F/CMR F could be a better choice for long-term treatments to avoid abnormal increase of spleen risk.

### Histopathological Studies (100× & Scale Bar 200 μm):

The IMQ induced, skin tissue sections (H&E stain) were collected after ten days (D1-D10) from all groups.

Normal Morphology (White Arrows): The histopathological sections clearly indicate that normal histopathological morphology was observed in the vehicle, disease and treated groups, respectively. Psoriatic indicators like acanthosis, hyperplasia, hyperkeratosis, and parakeratosis of the stratum corneal layer of the epidermis were absent in Metf+SMR F/CMR F treated groups. Dermal infiltration of granulocytes and lymphocytes may not be evident. Healthy morphological and histological features of skin were observed.

The above studies demonstrate that the formulations described in the present disclosure are effective in treating skin disorders and psoriasis

Thus, the present invention describes a pharmaceutical composition comprising metformin hydrochloride, silybin or curcumin, mangiferin, resveratrol, lemon grass oil, bakuchi oil. A topical hydrogel with the above composition was prepared to treat skin disorders and psoriasis. The formulation/composition also reduces one or more side effects associated with other treatments including corticosteroids. The pharmaceutical composition showed strong cytotoxicity in A431 cells indicating that it stops metastasis and psoriatic cell proliferation. A significant reduction of elevated inflammatory markers from Imiquimod-induced HaCat cells demonstrated that this composition worked in psoriasis.

### CONCLUSIONS

Following in vivo treatment with imiquimod-induced psoriasis mice model with this pharmaceutical composition, the body weight was significantly improved in diseased animals. Clinical Psoriasis Area and Severity Index (PASI) revealed that this pharmaceutical composition significantly reduced the Elevated Erythema, Skin Scaling, Epidermal thickness which describes that the present disclosure is effective in treating patients suffering from skin disorders and psoriasis. The combination of metformin and SMR or CMR formulation treatment showed an anti-psoriatic effect by decreasing epidermal phenotypic markers like acanthosis, hyperplasia, hyperkeratosis, and parakeratosis significantly. Further, these formulations reduced dermal infiltration of cytokines, T cells and keratinocyte proliferation more effectively which was equivalent to standard clobetasol formulation. So it is an effective anti-psoriatic formulation.

1. A composition comprising silybin or curcumin and biguanide or biguanide derivatives or biguanide salts.

2. The composition of claim 1, wherein the biguanide derivative is selected from a group comprising metformin, phenformin or buformin; biguanide salt is metformin hydrochloride.

3. The composition of claim 1, wherein the composition further comprises therapeutic molecule(s), oils, hydrogel/cream/ointment-base and pharmaceutical excipient(s), wherein the therapeutic molecule(s) is mangiferin and/or resveratrol, the oil is selected from a group comprising lemon grass oil, cream/ointment, sandalwood oil or bakuchi oil and combinations thereof, wherein the pharmaceutical excipients are selected from a group comprising thickener(s), preservative(s), alcohol(s), emollient(s), flavouring agent(s), surfactant(s), water and combinations thereof.

4. The composition of claim 1, comprising mangiferin, resveratrol, lemon grass oil, bakuchi oil and a hydrogel/cream/ointment base-excipients mixture and wherein the biguanide salt is metformin hydrochloride.

5. The composition of claim 4, wherein the hydrogel/cream/ointment base comprises crosslinked polyacrylic acid polymer, Triethanolamine, and water.

6. The composition of claim 5, wherein the hydrogel/cream/ointment base-excipients mixture comprises thickener(s), preservative(s), alcohol(s), emollient(s), flavouring agent(s), surfactant(s) and water.

7. The composition as claimed in claim 6, wherein
the thickener(s) is either Aloe vera and/or crosslinked polyacrylic acid polymer having viscosity 940;
the preservative(s) is EDTA and/or parabens,
the alcohol(s) is selected from a group comprising n-propanol, ethyl Alcohol, iso-propyl-alcohol, propylene glycol and PEGs;
the emollient(s) is selected from a group comprising propylene glycol, lanolin, emu oil, cocoa butter, ammonium lactate and fat soluble vitamins;
the flavouring agent(s) is selected from a group comprising lemon grass oil, sandalwood oil, emu oil and bakuchi oil; and
the surfactant(s) is selected from a group comprising Propylene glycol caprylate sodium laurylsulphate, Cetyltrimethylammonium bromide, Spans, Tweens, N-dodecyl-N, N-dimethyl betaine, and Labrasol.

8. The composition of claim 4, wherein
metformin hydrochloride is present at a concentration ranging from about 0.5% wt/wt to 1.5% wt/wt;
silybin is present at a concentration ranging from about 0.5% wt/wt to 1.5% wt/wt; or
curcumin is present at a concentration ranging from about 0.01% wt/wt to 0.1% wt/wt;
mangiferin is present at a concentration ranging from about 0.01% wt/wt to 0.1% wt/wt;
resveratrol is present at a concentration ranging from about 0.1% wt/wt to 1% wt/wt;
bakuchi oil is present at a concentration ranging from about 0.1% wt/wt to 0.2% wt/wt; and
hydrogel/cream/ointment base-excipients mixture ranges at a concentration ranging about 90% wt/wt to 98% wt/wt.

9. The composition of claim 8, wherein the hydrogel/cream/ointment base comprises
lemon grass oil at a concentration ranging from about 0.1% wt/wt to 1% wt/wt;
bakuchi oil at a concentration ranging from about 0.1% wt/wt to 0.2% wt/wt,
Aloe vera at a concentration of about 1% wt/wt;
Ethylenediamine tetraacetic acid at a concentration of about 0.1% wt/wt;
Isopropyl alcohol at a concentration of about 10% wt/wt;
Polyethylene glycol 400 at a concentration of about 10% wt/wt;
Propylene glycol caprylate at a concentration of about 1.5% wt/wt; and
crosslinked polyacrylic acid polymer having viscosity 940 at a concentration of about 1.5% wt/wt.

10. The composition of claim 1, wherein silybin (or) curcumin at is present a concentration of about 1% wt/wt or 0.05% (w/w), metformin hydrochloride is present at a concentration of about 1% wt/wt, mangiferin is present at a concentration of about 0.05% wt/wt, resveratrol is present at a concentration of about 0.5% wt/wt, lemon grass oil is present at a concentration of about 0.5% wt/wt, bakuchi oil is present at a concentration of about 0.16% wt/wt, Aloe vera is present at a concentration of about 1% wt/wt, Ethylenediamine tetra acetic acid is present at a concentration of about 0.1% wt/wt, Isopropyl alcohol is present at a concentration of about 10% wt/wt, Polyethylene glycol 400 is present at a concentration of about 10% wt/wt, Propylene glycol caprylate is present at a concentration of about 1.5% wt/wt, crosslinked polyacrylic acid polymer having a viscosity 940 is present at a concentration of about 1.5% wt/wt, and the hydrogel base-excipients mixture is present at a concentration of about 71.69% wt/wt, and the total parts of the components in the composition add up to 100% w/w.

11. (canceled)

12. The composition of claim 1, wherein the composition is in the form of a cream, ointment or gel.

13. A method of treating a patient afflicted with skin disorders, said method comprising topically administering a therapeutically effective amount of the composition as claimed in claim 1 to the patient.

14. The method of claim 13, wherein the skin disorders are selected from the group consisting of atopic dermatitis, seborrheic dermatitis, vitiligo, acne, psoriasis, and eczema.
